(19) United States
(12) Patent Application Publication
(10) Pub. No.: US 2026/0356871 A1
(43) Pub. Date: Nov. 12, 2026

(54) METHOD FOR PRODUCING SUGAR

(71) Applicant: THE UNIVERSITY OF OSAKA, Suita-shi, Osaka (JP)

(72) Inventors: Shuji NAKANISHI, Suita-shi (JP); Genta CHIKATANI, Suita-shi (JP); Hiroaki NISHIJIMA, Suita-shi (JP); Hiro TABATA, Suita-shi (JP); Yoko HASE, Nagakute-shi (JP)

(73) Assignee: THE UNIVERSITY OF OSAKA, Suita-shi, Osaka (JP)

(21) Appl. No.: 19/515,876

(22) PCT Filed: Aug. 29, 2024
(86) PCT No.: PCT/JP2024/031084

§ 371 Date: Mar. 3, 2026

(30) Foreign Application Priority Data
Sep. 4, 2023 (JP) 2023-143251

(51) Int. Cl. C07H 3/02; B01J 21/04; C25B 3/07; C25B 3/26

(52) U.S. Cl. CPC C07H 3/02; B01J 21/04; C25B 3/07; C25B 3/26

(57) ABSTRACT

The purpose of the present invention is to provide a method that, in a formose reaction which uses formaldehyde as a main raw material, can improve the efficiency of synthesis of sugar by increasing the reaction rate. Provided is a method for producing sugar, the method comprising a step in which formaldehyde is used as a substrate, sugar is used as an initiator, and an oxide containing aluminum as cations is caused to act as a catalyst in a buffer solution that has an effective buffer region at a pH of 6-9.

### TECHNICAL FIELD

The present invention relates to a method for producing a sugar by a selective formose reaction in which the reaction rate is improved.

### BACKGROUND ART

It is known that sugars are formed when an aqueous formaldehyde solution is heated in the presence of an appropriate catalyst. This reaction is called the formose reaction, and it has attracted interest from the viewpoints such as organic matter upgrading and the origin of life.

The formose reaction pathway includes a plurality of autocatalytic cycles as shown in FIG. 1. In the cycle of FIG. 1, the process begins with the formation of glycolaldehyde by the extremely slow dimerization of formaldehyde, and various sugar products are obtained through successive cross-aldol reactions and isomerization reactions between aldoses and ketoses. In addition, a retro-aldol reaction (C—C bond cleavage) of erythrose (C4) regenerates two molecules of glycolaldehyde, forming an autocatalytic cycle. In this manner, in the formose reaction, once a small amount of glycolaldehyde is formed from formaldehyde, sugar formation proceeds continuously through the regeneration of C2 or higher intermediates, which can become enolates, by the autocatalytic cycle.

In the formose reaction, under basic conditions, OH promotes the Cannizzaro reaction, which is a disproportionation reaction of aldehydes, resulting in low product selectivity, whereas under neutral conditions, the Cannizzaro reaction as a side reaction that occurs under basic conditions is expected to be suppressed to some extent.

As an example of a method for proceeding with the formose reaction under neutral conditions, NPL 1 reports that erythrulose and 3-pentulose are selectively obtained by inducing an aldol reaction between formaldehyde and dihydroxyacetone. In addition, as another example of the method for proceeding with the formose reaction under neutral conditions, NPL 2 reports that an aldol reaction between glycolaldehyde and formaldehyde proceeds by using hydroxyapatite as a catalyst, and sugar products including ribose are obtained.

NPL 1 and NPL 2 require the use of high-cost substances such as dihydroxyacetone or glycolaldehyde as starting substrates. Accordingly, PTL 1 discloses that the formose reaction can be proceeded using inexpensive formaldehyde as the main raw material by using, as a catalyst, a compound having the ability to deprotonate the hydroxyl group of erythrose but not having the ability to deprotonate water, and by using a sugar as an initiator. In addition, NPL 3 discloses that sugar synthesis by the formose reaction proceeds by using aluminum oxide as a catalyst.

### CITATION LIST

### Patent Literature

PTL 1: WO 2023/063364 A

### Non Patent Literature

NPL 1: Kinet. Catal. 2007, 48, 550-555
NPL 2: Org. Biomol. Chem. 2017, 15, 8888-8893
NPL 3: J. Mol. Evol. 1993, 36 (2), 101-106

### SUMMARY OF INVENTION

### Technical Problem

In the method described in PTL 1 and the method described in NPL 3, when aluminum oxide is used as a catalyst, the selectivity of the formose reaction can be improved using inexpensive formaldehyde as the main raw material, and thus the method is excellent in usefulness from the viewpoint of carbon upgrading. On the other hand, from the viewpoint of reaction rate, there is still room for improvement. Accordingly, an object of the present invention is to provide a method capable of improving the efficiency of sugar synthesis by increasing the reaction rate of the formose reaction using formaldehyde as the main raw material.

### Solution to Problem

As a result of extensive investigation, the present inventor has found that the reaction rate of the formose reaction is improved by causing the formose reaction in which formaldehyde is used as a substrate, sugar is used as an initiator, and an oxide containing aluminum as cations is used to act as a catalyst to proceed in a buffer solution having an effective buffer region at a pH of 6 to 9.

That is, the present invention provides the inventions according to the following aspects.

Item 1. A method for producing a sugar, including a step in which formaldehyde is used as a substrate, sugar is used as an initiator, and an oxide containing aluminum as cations is caused to act as a catalyst in a buffer solution that has an effective buffer region at a pH of 6 to 9.

Item 2. The method for producing a sugar according to Item 1, in which the buffer solution is selected from the group consisting of phosphate buffer solution, Good's buffer solution, citrate buffer solution, Tris buffer solution, citrate-phosphate buffer solution, and McIlvaine buffer solution.

Item 3. The method for producing a sugar according to Item 1 or 2, in which the concentration of the buffering agent in the buffer solution is 0.01 to 0.5 M in a reaction mixture containing the substrate, the initiator, the catalyst, and the buffer solution.

Item 4. The method for producing a sugar according to any one of Items 1 to 3, in which the concentration of the buffering agent in the buffer solution is 0.02 to 1 times the concentration of the catalyst on a molar basis in a reaction mixture containing the substrate, the initiator, the catalyst, and the buffer solution.

Item 5. The method for producing a sugar according to any one of Items 1 to 4, in which the catalyst is aluminum oxide.

Item 6. The method for producing a sugar according to any one of Items 1 to 5, in which the initiator is used in a ratio of 0.1 to 50 millimoles relative to 1 mole of the substrate.

Item 7. The method for producing a sugar according to any one of Items 1 to 6, further including
a step of obtaining the formaldehyde by electrolysis of carbon dioxide, in which
the step of obtaining the formaldehyde and the step of causing the catalyst to act are carried out in the electrolysis system.

Item 8. A sugar production system including a carbon dioxide electrolysis device for obtaining formaldehyde from carbon dioxide, a catalyst composed of an oxide containing aluminum as cations, and a buffer solution having an effective buffer region at pH 6 to 9.

### Advantageous Effects of Invention

According to the present invention, it is possible to improve the efficiency of sugar synthesis by increasing the reaction rate of the formose reaction using formaldehyde as the main raw material.

### BRIEF DESCRIPTION OF DRAWINGS

FIG. 1 shows a formose reaction pathway.

FIG. 2A shows formaldehyde concentrations after a formose reaction was performed at 80 °C for 3 days in Comparative Example 1 and Comparative Example 2.

FIG. 2B shows chromatograms of product distributions of Comparative Example 1 and Comparative Example 2 after the formose reaction was performed at 80 °C for 3 days.

FIG. 3 shows temporal changes in formaldehyde concentration in a reaction system in Example 1, Comparative Example 1, and Comparative Example 3.

FIG. 4A shows pH changes of reaction solutions in Example 1 and Comparative Example 3 together with pH changes of reaction solutions in Comparative Example 1.

FIG. 4B shows respective concentration changes of HPO₄²⁻ and H₂PO⁴⁻ in the reaction solutions in Example 1 and Comparative Example 3.

FIG. 5 shows chromatograms of product distributions according to Example 1 and Comparative Example 1.

FIG. 6 shows formaldehyde (HCHO) concentrations 72 hours after the start of the reaction according to Example 2 and Comparative Example 1.

### DESCRIPTION OF EMBODIMENTS

### 1. Method for Producing Sugar

A method for producing a sugar of the present invention includes a step (hereinafter also referred to as the “formose reaction step”) in which formaldehyde is used as a substrate, sugar is used as an initiator, and an oxide containing aluminum as cations is caused to act as a catalyst in a buffer solution that has an effective buffer region at a pH of 6 to 9. The method for producing a sugar of the present invention constructs a formose reaction system exemplified in FIG. 1 to produce a sugar.

In the formose reaction step, a reaction mixture containing formaldehyde, a sugar, a predetermined catalyst, and a predetermined buffer solution is subjected to reaction conditions that proceed the formose reaction, thereby producing a sugar in the reaction mixture. The formose reaction may be carried out by a batch method or by a flow method. In the case of the batch method, formaldehyde, a sugar, a predetermined catalyst, and a predetermined buffer solution can be added to a reaction vessel, mixed, and reacted. In the case of the flow method, a reaction mixture other than the catalyst can be caused to flow through the column on which the catalyst is immobilized to perform the reaction. In the case of the flow method, a reaction mixture containing formaldehyde, a sugar, a predetermined catalyst, and a predetermined buffer solution is formed within the column.

### 1-1. Substrate

The amount of formaldehyde, which serves as the substrate, to be incorporated into the reaction mixture is not particularly limited, but may be, for example, 0.01 to 5 M, preferably 0.05 to 3 M, more preferably 0.1 to 1 M, and still more preferably 0.2 to 0.5 M.

### 1-2. Initiator

The sugar used as an initiator in the present invention includes monosaccharides, disaccharides, oligosaccharides, and polysaccharides.

The term “monosaccharide” refers to monosaccharides including diose, and specifically includes monosaccharides having 2 to 7 carbon atoms (that is, diose, triose, tetrose, pentose, hexose, and heptose). The monosaccharide may be either an aldose or a ketose. Furthermore, the monosaccharide may have a chain (linear or branched) structure or a cyclic structure.

Specific examples of monosaccharides include glycolaldehyde, dihydroxyacetone, glyceraldehyde, erythrose, erythrulose, ribose, arabinose, xylose, lyxose, ribulose, xylulose, psicose, fructose, sorbose, tagatose, allose, altrose, glucose, mannose, gulose, idose, galactose, talose, and heptulose. These monosaccharides may be used alone or in combination of two or more kinds thereof. Among these monosaccharides, glycolaldehyde is preferable.

A disaccharide is a compound in which two monosaccharides are condensed. Specific examples of disaccharides include lactose, maltose, isomaltose, sucrose, and trehalose. These disaccharides may be used alone or in combination of two or more kinds thereof.

An oligosaccharide is a compound in which 3 to 10 monosaccharides are condensed. Specific examples of oligosaccharides include fructooligosaccharides, galactooligosaccharides, xylooligosaccharides, soybean oligosaccharides, isomaltooligosaccharides, lactosucrose, and raffinose. These oligosaccharides may be used alone or in combination of two or more kinds thereof.

A polysaccharide is a compound in which 11 or more monosaccharides are condensed. Specific examples of polysaccharides include starch, dextrin, cellulose, glycogen, dextran, mutan, levan, and inulin. These polysaccharides may be used alone or in combination of two or more kinds thereof.

In the present invention, the sugar may be used in any one of the four forms of sugar including monosaccharide, disaccharide, oligosaccharide, and polysaccharide, or in a combination of two or more kinds thereof. Among these sugars, from the viewpoint of improving the reaction efficiency of the formose reaction and/or the selectivity of the formose reaction, monosaccharides, disaccharides, and oligosaccharides are preferable. More preferably, monosaccharides and disaccharides are used, still more preferably, monosaccharides are used; and particularly preferably, glycolaldehyde is used.

In the production method of the present invention, sugar formation proceeds continuously based on the promotion of regeneration of intermediate products serving as nucleophiles through promotion of the retro-aldol reaction in the formose reaction, and therefore, the amount of sugar to be included in the reaction mixture may be only an amount sufficient as an initiator. The amount of initiator used in the production method of the present invention is not particularly limited, but may be, for example, 0.1 to 50 millimoles, preferably 3 to 30 millimoles, more preferably 5 to 15 millimoles, and still more preferably 8 to 12 millimoles, per mole of the charged formaldehyde.

### 1-3. Catalyst

The predetermined catalyst used in the production method of the present invention is an oxide containing aluminum as cations. Since the oxide containing aluminum as cations has basic catalytic activity, it becomes possible to produce a sugar using formaldehyde as a substrate by using the oxide together with a sugar as an initiator.

The oxide containing aluminum as cations is not particularly limited as long as at least part of the cations constituting the oxide is aluminum, the cations may consist solely of aluminum, or may include, in addition to aluminum, other cations such as sodium, silicon, and/or zirconium. A preferable example of the oxide containing aluminum as cations is aluminum oxide (Al₂O₃).

The amount of catalyst used is not particularly limited, but may be, for example, 0.1 to 40 moles per mole of the charged formaldehyde. When the reaction is carried out by the batch method, the amount of catalyst per mole of the charged formaldehyde may be, for example, 0.1 to 5 moles, preferably 0.5 to 3 moles, more preferably 1 to 2 moles, and still more preferably 1.2 to 1.8 moles. When the reaction is carried out by the flow method, the amount of catalyst per mole of the charged formaldehyde may be, for example, 10 to 40 moles, and preferably 30 to 38 moles.

### 1-4. Buffer Solution

In the production method of the present invention, in order to increase the reaction rate of the formose reaction, a buffer solution having an effective buffer region at pH 6 to 9 (that is, a buffer solution prepared such that the effective buffer region falls within pH 6 to 9) is used. The pH refers to a pH at 25 °C.

The buffer solution may be any buffer solution prepared such that the effective buffer region falls within pH 6 to 9. Preferable examples of such effective buffer solutions include those having an effective buffer region of pH 6.2 to 8, more preferably 6.4 to 7.5, and still more preferably 6.6 to 7.2. It is to be noted that, since the concentration of ions constituting the buffer solution varies as the formose reaction proceeds, deviation of the pH of the reaction mixture from the above effective buffer region during the course of the formose reaction is allowed.

The type of buffer solution is not particularly limited as long as the buffer solution has the above effective buffer region, and may be an aqueous solution of a weak acid and the conjugate base, or an aqueous solution of a weak base and the conjugate acid. It is to be noted that, hereinafter, the mixture of a weak acid and the conjugate base or the mixture of a weak base and the conjugate acid, which serves as the solute of the buffer solution, will be referred to as the buffering agent.

Specific examples of the buffer solution include, for example, phosphate buffer solution, Good's buffer solution, citrate buffer solution, Tris buffer solution, citrate-phosphate buffer solution, and Mcllvaine buffer solution. Among these buffer solutions, phosphate buffer solution and Good's buffer solution (more preferably MOPS buffer solution) are preferable, and phosphate buffer solution is still more preferable.

The concentration of the buffer solution in the reaction mixture may be appropriately determined according to the desired degree of improvement in the reaction rate of the formose reaction. Specific examples of the concentration of the buffer solution in the reaction mixture, in terms of buffering agent concentration, include, for example, 0.01 to 0.5 M, preferably 0.02 to 0.3 M, more preferably 0.03 to 0.2 M, still more preferably 0.04 to 0.1 M, 0.04 to 0.08 M, or 0.04 to 0.06 M.

The ratio of the buffering agent concentration to the amount of catalyst may also be appropriately determined according to the desired degree of improvement in the reaction rate of the formose reaction. Specific examples of the ratio of the buffering agent concentration to the catalyst concentration (on a molar basis) include, for example, 0.02 to 1 times, preferably 0.035 to 0.7 times, more preferably 0.05 to 0.4 times, still more preferably 0.07 to 0.2 times, and even more preferably 0.09 to 0.15 times or 0.09 to 0.13 times of the catalyst.

### 1-5. pH Conditions

In the production method of the present invention, the reaction mixture is subjected to pH conditions encompassing the above effective buffer region, and the formose reaction is allowed to proceed. The pH of the reaction mixture at the start of the reaction (referring to the pH at 25 °C) is preferably pH 6 to 9, more preferably 6.2 to 8.6, still more preferably 6.4 to 8.4, even more preferably 6.6 to 8.4, 7 to 8.4, or 7.5 to 8.4.

### 1-6. Other Reaction Conditions and the Like

In the production method of the present invention, sugar formation proceeds continuously based on the promotion of regeneration of intermediates through promotion of the retro-aldol reaction in the formose reaction, and therefore, it is not necessary to add substrates other than formaldehyde to the reaction mixture. Therefore, in one embodiment of the production method of the present invention, no compounds other than formaldehyde are added to the reaction mixture as substrates (specifically, this means that the intermediates shown in FIG. 1 are not added as substrates, which is to be distinguished from the use of a sugar as an initiator as described above). It should be noted that the present invention does not exclude forms in which substrates other than formaldehyde are added, and in another embodiment, substrates other than formaldehyde are added.

Examples of the reaction solvent used in the reaction mixture include at least water, and from the viewpoint of suppressing side reactions, a mixed solution of water and a lower alcohol is preferable. Examples of lower alcohols include monohydric lower alcohols having 1 to 4 carbon atoms, preferably methanol, ethanol, and isopropyl alcohol, more preferably methanol and ethanol, and still more preferably methanol. The amount of the lower alcohol contained in the mixed solution of water and the lower alcohol is, for example, 1 to 20 vol %, preferably 5 to 15 vol %, and more preferably 8 to 12 vol % from the viewpoint of suppressing side reactions.

The reaction temperature to which the reaction mixture is subjected is not particularly limited as long as the formose reaction can proceed, but may be, for example, 50 to 100 °C, preferably 70 to 90 °C, and more preferably 75 to 85 °C. The reaction time may be appropriately determined depending on factors such as the scale of the reaction mixture and the amount of sugar formed, but may be, for example, 12 to 84 hours, and preferably 24 to 72 hours.

### 1-7. Other Steps

The production method of the present invention can be combined with an electrochemical reaction that produces formaldehyde by electrochemical reduction of carbon dioxide. That is, in this case, the production method of the present invention further includes a step of obtaining the formaldehyde, which is the substrate of the above-described step, by electrolysis of carbon dioxide (hereinafter also referred to as the “electrolysis reaction step”), and the electrolysis reaction step and the above-described formose reaction step can be carried out within the electrolysis reaction system.

Normally, since an aqueous solution of carbon dioxide is acidic to neutral, whereas the conventional formose reaction using formaldehyde as a substrate proceeds under basic conditions, the formose reaction cannot be carried out in the electrolysis reaction solution (that is, within the same reaction system). On the other hand, in the production method of the present invention, since the formose reaction step can proceed under neutral conditions, the electrolysis reaction step and the formose reaction step can be performed in the same reaction system, that is, in the electrolysis reaction system.

After completion of the reaction, the reaction mixture is cooled, and the formed sugar can be purified by a known method. Examples of purification methods include cation exchange chromatography, anion exchange chromatography, and silica gel column chromatography, which may be used alone or in combination of two or more thereof, depending on the components to be removed.

### 1-8. Sugar to be Produced

The sugar to be produced in the method of the present invention typically includes pentoses and/or hexoses. Specific examples of the sugar to be produced in the method of the present invention include 1,3,4-trihydroxy-3-(hydroxymethyl) butan-2-one, 1,3,4,5-tetrahydroxypentan-2-one, 1,2,4,5,6-pentahydroxyhexan-3-one, glucose, sorbose, fructose, mannose, and galactose.

### 2. Sugar Production System

As described above, since the oxide containing aluminum as cations and the buffer solution having an effective buffer region at pH 6 to 9 can promote the formose reaction, by combining the electrolysis reaction of carbon dioxide to formaldehyde in the above-described sugar production method, it becomes possible to carry out the electrolysis reaction and the formose reaction within the same reaction system, that is, within the electrolysis reaction system. Therefore, the present invention includes a sugar production apparatus including a carbon dioxide electrolysis device for obtaining formaldehyde from carbon dioxide, an oxide containing aluminum as cations, and a buffer solution having an effective buffer region at pH 6 to 9.

A carbon dioxide electrolysis device for obtaining formaldehyde from carbon dioxide is well known to those skilled in the art, and any carbon dioxide electrolysis device is appropriately selected by those skilled in the art. In addition, the catalyst (oxide containing aluminum as a cation) and the buffer solution are provided in any aspect capable of contacting with formaldehyde generated by the carbon dioxide decomposer. Details of the catalyst and the buffer solution are as described above in “1. Method for Producing Sugar”. The sugar production apparatus of the present invention is used for carrying out the production method according to an aspect combined with an electrochemical reaction for producing formaldehyde by electrochemical reduction of carbon dioxide among the aspects described in “1. Method for Producing Sugar” above.

### EXAMPLES

Hereinafter, the present invention will be described more specifically with reference to Examples, but the present invention is not limited thereto.

### [Reagents and Apparatus]

The reagents and apparatus used in the following test examples are as follows.

### <Catalyst>

Aluminum oxide Al₂O₃ (sigma)

### <Buffer Solution>

0.1 M phosphate buffer solution (pH 7.0 (25 °C)): Prepared by mixing 6.10 mL of 0.2 M Na₂HPO₄ aqueous solution, 3.90 mL of 0.2 M NaH₂PO₄ aqueous solution, and 10 mL of ultrapure water.
0.1 M MOPS buffer solution (pH 6.5 to 7.9 (25 °C)): Prepared by diluting 1 M MOPS buffer solution (purchased from DOJINDO LABORATORIES) tenfold with ultrapure water.

### <Comparative Solution>

NaH₂PO₄ aqueous solution (0.1M)

### <Initiator>

Glycolaldehyde (Trade name: Glycolaldehyde dimer) (sigma aldrich)

### <Substrate>

Formaldehyde solution (wako)

### <Others>

Methanol (wako)
2,4-Dinitrophenylhydrazine (Sigma-Aldrich)
Acetonitrile for high-performance liquid chromatography (HPLC) (wako)
Anion exchange resin AmberLite IRIRA402BL Cl (Organo Corporation)
Syringe filter (PTFE013022L AS ONE)
Centrifuge (MX-305 TOMY SEIKO CO., LTD.)
10 mL screw vial (Nippon Electric Glass Co., Ltd)
Organic synthetic stirrer (HHE-19G-USIV KPI)
High-performance liquid chromatograph (Hitachi, Ltd.)
pH meter (D-210P Horiba)

### Test Example 1

The formose reaction was carried out under the following conditions.

TABLE 1

| | | Comparative | Comparative | Comparative |
|---|---|---|---|---|
| | Example 1 | Example 1 | Example 2 | Example 3 |
| Solvent | 0.05M phosphate | 10% v/v aqueous | 0.05M phosphate | 0.05M NaH₂PO₄ |
| | buffer solution + | methanol | buffer solution + | aqueous solution + |
| | 10% v/v aqueous | | 10% v/v aqueous | 10% v/v aqueous |
| | methanol | | methanol | methanol |
| Catalyst | 50 mg/mL | 50 mg/mL | | 50 mg/mL |
| | Aluminum oxide | Aluminum oxide | | Aluminum oxide |
| Initiator | 3 mM | 3 mM | 3 mM | 3 mM |
| | Glycolaldehyde | Glycolaldehyde | Glycolaldehyde | Glycolaldehyde |
| Substrate | 0.3M | 0.3M | 0.3M | 0.3M |
| | Formaldehyde | Formaldehyde | Formaldehyde | Formaldehyde |

aq: Aqueous solution

### <Preparation of Reaction Solution>

The solution used for Example 1 and Comparative Example 2 (formose reaction in phosphate buffer solution) was prepared by mixing 0.1 M phosphate buffer solution and a 20 vol % aqueous methanol solution containing 0.6 M formaldehyde and 6 mM glycolaldehyde at a volume ratio of 1:1. The solution used for Comparative Example 3 (formose reaction in NaH₂PO₄ aqueous solution) was prepared by mixing 0.1 M NaH₂PO₄ aqueous solution and a 20 vol % aqueous methanol solution containing 0.6 M formaldehyde and 6 mM glycolaldehyde at a volume ratio of 1:1. As the solution used for Comparative Example 1 (other formose reaction), a 10 vol % aqueous methanol solution containing 0.3 M formaldehyde and 3 mM glycolaldehyde was used.

### <Formose Reaction>

The formose reaction was carried out by the following operations.

Aluminum oxide was added to the above reaction solution at 50 mg (0.49 mmol) per 1 mL, and a reaction mixture with a total volume of approximately 2 mL was prepared. The prepared mixture was placed together with a stir bar into a screw vial (Nichiden-Rika Glass Co., Ltd.), and the formose reaction was carried out by stirring and heating at 80 °C using an organic synthesis stirrer (KPI). After completion of the reaction, the mixture was cooled with ice water, separated using a centrifuge (TOMY SEIKO CO., LTD.), and then filtered through a syringe filter (AS ONE) to remove the aluminum oxide. Thereafter, phosphate ions were removed using an anion exchange resin (Organo Corporation), and the formed mixture was obtained.

### <DNPH Derivatization>

The formed mixture was DNPH derivatized as follows and prepared as a sample for analysis.

2.5 μL of the formed mixture was dissolved in 748 μL of ultrapure water. 375 μL of an acetonitrile solution (1 mg/l mL) of 2,4-dinitrophenylhydrazine (DNPH) was added thereto, and 22.5 μL of a 20% aqueous phosphoric acid solution was added thereto. Thereafter, the mixture was stirred using a stirrer for 60 minutes to obtain a sample for analysis.

### <Analysis by HPLC>

Formaldehyde and the formose reaction intermediate up to a tetrose were analyzed by subjecting the DNPH-derivatized sample for analysis to HPLC as described above. A high-performance liquid chromatograph (Hitachi, Ltd.) was used as an apparatus, and a UV/Vis detector (Hitachi, Ltd.) was used as a detector. Inertsustain C18 (GL Sciences) was used for the column. As a mobile phase, a mixed solution of acetonitrile for high-performance liquid chromatograph (FUJIFILM Wako) and ultrapure water 4:6 (volume ratio) was used, and a flow rate was set to 1.0 mL/min.

The analysis of pentoses and hexoses was performed by diluting the formed mixture with an equal volume of acetonitrile and subjecting the mixture to HPLC. The apparatus used was the Chromaster® sugar (phosphate-phenylhydrazine method) analysis system for high-performance liquid chromatography (Hitachi, Ltd.). NH2P-50 4E (Shodex) was used as a column. As the mobile phase, a gradient flow of acetonitrile for high-performance liquid chromatography (FUJIFILM Wako), ultrapure water, and 10% aqueous phosphoric acid solution was used, with a flow rate of 1.0 mL/min.

### <Results>

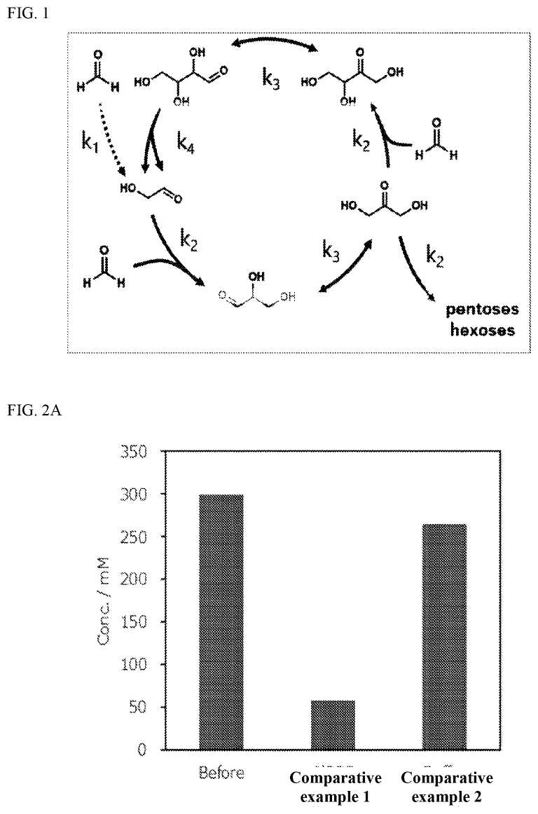

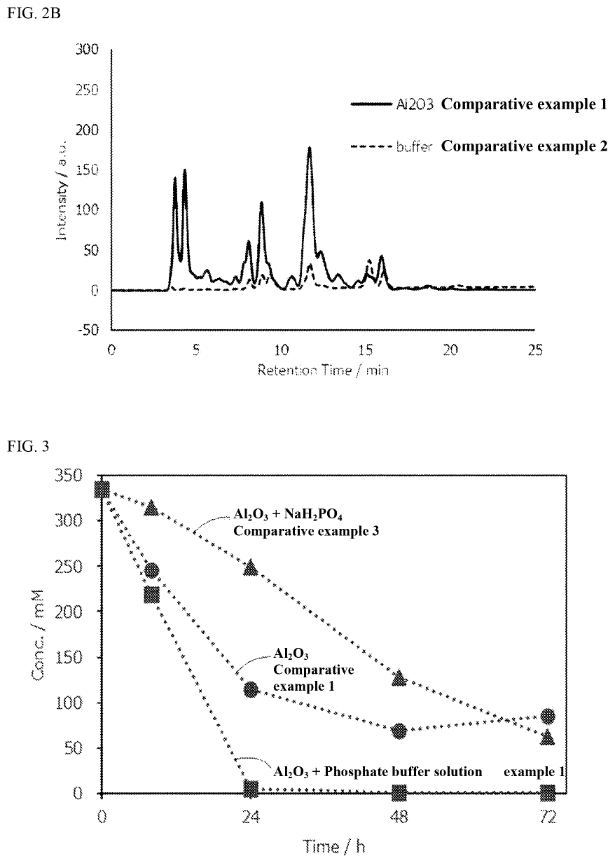

(1) For Comparative Example 1 and Comparative Example 2, the formaldehyde concentration and product distribution after carrying out the formose reaction at 80 °C for 3 days are shown in FIGS. 2A and 2B, respectively. As shown in the results of Comparative Example 1, an autocatalytic cycle is formed in the formose reaction system by the aluminum oxide catalyst, resulting in the consumption of formaldehyde in an amount exceeding that of the added initiator, and the formation of a large amount of sugar products. On the other hand, as shown in the results of Comparative Example 2, since the retro-aldol reaction does not proceed in the phosphate buffer solution, an autocatalytic cycle is not formed, only the amount of formaldehyde corresponding to the added initiator was consumed, and only a small amount of sugar products was formed.

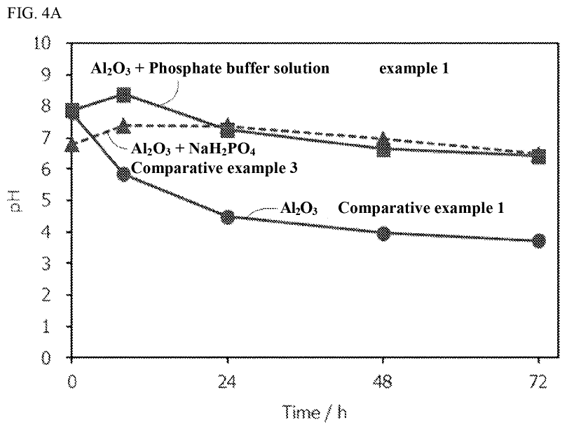

(2) The time-dependent change in the formaldehyde concentration in the reaction system in Example 1, Comparative Example 1, and Comparative Example 3 is shown in FIG. 3. As shown in FIG. 3, in the reaction system in which phosphate buffer solution was added together with aluminum oxide (Example 1), the consumption of formaldehyde was accelerated, and almost all of the formaldehyde was consumed. On the other hand, it has been found that in the reaction system (Comparative Example 3) in which a NaH₂PO₄ solution having a pH equivalent to that of the phosphate buffer solution is added together with aluminum oxide, the consumption of formaldehyde is remarkably slow, and the rate thereof is slower than the consumption rate of formaldehyde in the reaction system (Comparative Example 1) in which only aluminum oxide is added.

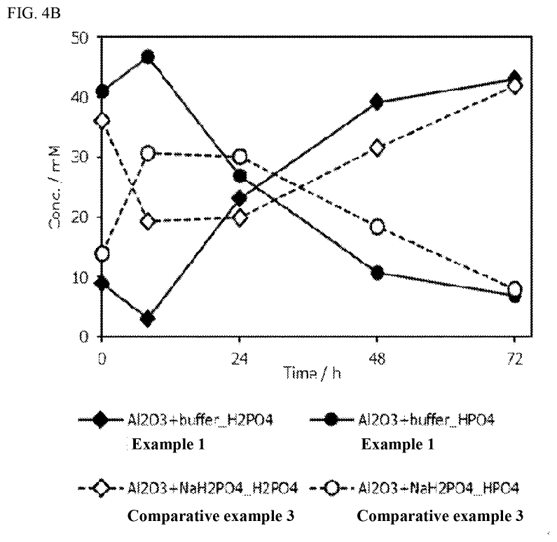

(3) In order to clarify the factor of the difference in reaction activity between Example 1 and Comparative Example 3, which was confirmed in FIG. 3, the change in pH (in terms of pH at 25 °C) of the reaction solution and the concentration change of each of HPO₄²⁻ and H₂PO⁴⁻ in the reaction solution calculated from the pKa value of the phosphate ion are shown in FIGS. 4(A) and 4(B), respectively. As shown in FIG. 4(A), there was almost no difference in pH change between Example 1 and Comparative Example 3. As shown in FIG. 4(B), during 0 to 24 hours after the start of the reaction (that is, the time period in which there was a particularly notable difference in reaction activity as shown in FIG. 3), it was found that the concentration of HPO₄²⁻ ions in Example 1 was higher than that in Comparative Example 3. Therefore, in Example 1, it is considered that the strongly basic HPO₄²⁻ ions effectively functioned as a base catalyst and contributed to the increase in the formaldehyde consumption rate.

(4) FIG. 5 shows chromatograms of the product distribution in Example 1 and Comparative Example 1 (both at 66.5 hours after the start of the reaction). As compared with Comparative Example 1, in Example 1, the peak before the retention time of 5 minutes was suppressed, and the peak after the retention time of 10 minutes was increased. That is, in Example 1, it was also recognized that the distribution of the product can be simplified (the product selectivity can be improved). The sugars observed in Example 1 included 1,3,4-trihydroxy-3-(hydroxymethyl) butan-2-one, 1,3,4,5-tetrahydroxypentan-2-one, 1,2,4,5,6-pentahydroxyhexan-3-one, glucose, sorbose, fructose, mannose, and galactose. In particular, in Example 1, the formation of glucose that was not formed in Comparative Example 1 was clearly observed, and the production amount of sorbose was significantly increased as compared with Comparative Example 1.

(5) Based on the above results, it was found that by allowing aluminum oxide and phosphate buffer solution to act cooperatively, it was possible to synthesize sugars at a formaldehyde consumption rate higher than that achievable by aluminum oxide alone or phosphate buffer solution alone. Furthermore, it was also found that not only the reaction rate can be increased, but also the product selectivity can be improved.

### Test Example 2

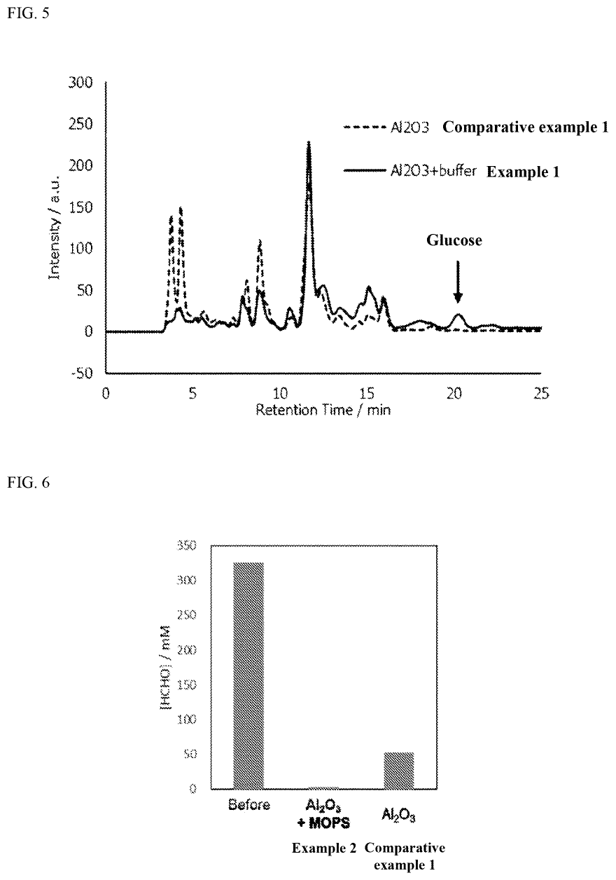

A formose reaction was carried out in the same manner as in Example 1 except that the buffer solution was replaced by a MOPS buffer solution (Example 2). The pH (in terms of pH at 25 °C) of the reaction mixture at the start of the reaction in Example 2 was 6.87. The formaldehyde (HCHO) concentration 72 hours after the start of the reaction is shown in FIG. 6. In view of the HCHO concentration before the reaction, the HCHO conversion rate in Comparative Example 1 using Al₂O₃ alone was 84%, whereas in Example 2, in which MOPS buffer solution was added, the HCHO conversion rate was improved to 99%. That is, even when a MOPS buffer solution was used as a buffer solution, an increase in the formose reaction rate was observed.

1. A method for producing a sugar, including a step in which formaldehyde is used as a substrate, sugar is used as an initiator, and an oxide containing aluminum as cations is caused to act as a catalyst in a buffer solution that has an effective buffer region at a pH of 6 to 9.

2. The method for producing a sugar according to claim 1, wherein the buffer solution is selected from the group consisting of phosphate buffer solution, Good's buffer solution, citrate buffer solution, Tris buffer solution, citrate-phosphate buffer solution, and McIlvaine buffer solution.

3. The method for producing a sugar according to claim 1, wherein the concentration of the buffering agent in the buffer solution is 0.01 to 0.5 M in a reaction mixture containing the substrate, the initiator, the catalyst, and the buffer solution.

4. The method for producing a sugar according to claim 1, wherein the concentration of the buffering agent in the buffer solution is 0.02 to 1 times the concentration of the catalyst on a molar basis in a reaction mixture containing the substrate, the initiator, the catalyst, and the buffer solution.

5. The method for producing a sugar according to claim 1, wherein the catalyst is aluminum oxide.

6. The method for producing a sugar according to claim 1, wherein the catalyst is used in a ratio of 0.1 to 5 moles relative to 1 mole of the substrate.

7. The method for producing a sugar according to claim 1, further comprising
a step of obtaining the formaldehyde by electrolysis of carbon dioxide, wherein
the step of obtaining the formaldehyde and the step of causing the catalyst to act are carried out in the electrolysis system.

8. A sugar production system including a carbon dioxide electrolysis device for obtaining formaldehyde from carbon dioxide, a catalyst composed of an oxide containing aluminum as cations, and a buffer solution having an effective buffer region at pH 6 to 9.
